(19) United States
(12) Patent Application Publication
(10) Pub. No.: US 2011/0053156 A1
(43) Pub. Date: Mar. 3, 2011

(54) SMALL CELL LUNG CARCINOMA BIOMARKER PANEL

(75) Inventors: Ann Vander Borght, Hasselt (BE); Franciscus Charles Servatius Ramaekers, Maastricht (NL); Stefan Maarten Van Den Eijnde, 's Gravenvoeren (BE); Marjan Harmsma, Eijsden (NL); Frank Walter Falkenberg, Dortmund (DE); Kaï Stühler, Bochum (DE); Gereon Poschmann, Bochum (DE); Helmut Erich Meyer, Bochum (DE); Günter Kloppel, München (DE)

(73) Assignee: MUBIO PRODUCTS BV, Maastricht (NL)

(21) Appl. No.: 12/865,406

(22) PCT Filed: Feb. 19, 2009
(86) PCT No.: PCT/EP09/01215

§ 371 Date: Oct. 20, 2010

(30) Foreign Application Priority Data
Feb. 22, 2008 (GB) 0803192.4

(51) Int. Cl. C12Q 1/68; G01N 33/574; C07K 14/435

(52) U.S. Cl. 435/6; 435/7.94; 435/7.1; 436/501; 530/357; 530/350

(57) ABSTRACT

The invention relates generally to the field of cancer detection, diagnosis, subtyping, staging, prognosis, treatment and prevention. More particularly, the present invention relates to methods for the detection, and/or diagnosing and/or subtyping and/or staging of lung cancer in a patient. Based on a particular panel of biomarkers, the present invention provides methods to detect, diagnose at an early stage and/or differentiate small cell lung cancer (SCLC) from non-small cell lung cancer (NSCLC) and within NSCLC to differentiate between squamous cell carcinomas (SCC), adenocarcinomas (AC), within SCC to discriminate G2 and G3 stage and within lung cancer to differentiate for lung cancers with or without neuroendocrine origin. It further provides the use of said panel of biomarkers in monitoring disease progression in a patient, including both in vitro and in vivo imaging techniques. The in vitro imaging techniques typically include an immunoassay detecting protein or antibody of the biomarkers on a sample taken from said patient, e.g. serum or tissue sample. The in vivo imaging techniques typically include chest radiographs (X-rays), Computed Tomography (CT) imaging, spiral CT, Positron Emission Tomography (PET), PET-CT and scintigraphy for molecular imaging and diagnosis and to monitor disease progression and treatment response in patients. It is accordingly a further aspect to provide a kit to perform the aforementioned diagnosing and/or subtyping and/or staging assay and the imaging techniques, comprising reagents to determine the gene expression or protein level of the aforementioned panel of biomarkers for in vitro and in vivo applications.

### FIELD OF THE INVENTION

The invention relates generally to the field of cancer diagnosis, prognosis, treatment and prevention. More particularly, the present invention relates to methods subtyping lung cancer in a patient. Based on a particular panel of biomarkers, the present invention provides methods to (early) diagnose lung cancer and methods to differentiate small cell lung cancer (SCLC) from non-small cell lung cancer (NSCLC) and within NSCLC to differentiate between squamous cell carcinomas (SCC), adenocarcinomas (AC) and eventually large cell carcinoma, a method to discriminate between lung cancers with or without neuroendocrine origin, a method to discriminate between G2 and G3 grade SCC tumors, and a method to determine the degree of heterogeneity of lung cancer.

It further provides the use of said panel of biomarkers in monitoring disease progression in a patient, including both in vitro and in vivo imaging techniques. The in vitro imaging techniques typically include an immunoassay on a sample taken from said patient, e.g. serum or tissue sample.
The in vivo imaging techniques typically include chest radiographs (X-rays), Computed Tomography (CT) imaging, spiral CT, Positron Emission Tomography (PET), PET-CT and scintigraphy.

It is accordingly a further aspect to provide a kit to perform the aforementioned imaging techniques, comprising reagents to determine the expression of the aforementioned panel of biomarkers. In particular comprising antibodies, specific for the panel of biomarkers identified hereinafter.

### BACKGROUND TO THE INVENTION

Lung cancer is one of the most frequent cancer types and additionally in both Europe and the US the main cause of cancer related mortality. In 2004 lung cancer was responsible for 20% of all cancer related cases of death in Europe and of 29% in the US (1, 2).

Lung cancer is generally categorized into two classes, small lung cancer carcinoma (SCLC), that accounts for about 15 to 20% of lung cancer patients, and non-small lung cancer carcinoma (NSCLC), that accounts for approximately 80 to 85% of all lung cancers and can be further subdivided into lung squamous cell carcinoma (SCC), lung adenocarcinoma (AC), and large cell carcinoma (LC).

The two main groups differ in both their growth and treatment characteristics. SCLC tumors exhibit an aggressive phenotype susceptible to chemo- and radiotherapy, whereas NSCLC are not chemosensitive and commonly treated by surgery. However, currently no adequate treatment protocols for the different types of lung cancer exist. With conventional therapy, median survival for the subtype of SCLC is 15 months for limited-stage disease and 9 months for extensive-stage disease, whereas long-term survival is very low. In view of the differences in behavior between the two main categories, the decision upon treatment protocols is especially guided by the subdivision into SCLC and NSCLC.

Unlike the other types of lung cancer, SCLC is sensitive to chemotherapy. In about 75% of the cases of SCLC an initial response to chemotherapy can be noticed, with a clinically complete response in about 35% of all cases (Johnson D H, et al., 1987; Am J Med Sci 293: 377-389). Unfortunately, however, in most cases relapse occurs, resulting in a three-year survival rate of only 5-10%, and a five-year survival rate of about 1% (Minna J D, et al. 1985, Cancer of the lung. In: Cancer. Principles and practice of oncology 2nd ed); Within SCLC a clinically relevant subdivision can be made between classic and variant SCLC. The variant-type of SCLC appears to be even less sensitive to chemotherapy and radiotherapy. As a result the median survival time of patients suffering from the variant-type of SCLC is significantly shorter than of those with a classic type of SCLC (Radice P A, et al. 1982, Cancer; 50: 2894-2902). Also for patients with a combined SCLC a poorer prognosis than for patients with classic SCLC is observed (Hirsch F R et al, 1983, Cancer; 52: 2144-2150). Approximately 75% to 80% of cases are of the NSCLC histology, and the majority of patients present with either locally advanced disease (stage III) or metastatic disease (stage 1V). Importantly, patients undergoing curative surgical resection for apparent localized disease have survival rates ranging between 50% and 80%, implying the need for better systemic treatment to cure occult micrometastatic disease. In NSCLC treatment with chemotherapy is in general unsuccessful (Minna J D, et al. 1985, Cancer of the lung. In: Cancer. Principles and practice of oncology; 2nd ed.). Therefore, with the exception of high cure rates for surgical treatment of truly localized disease, the prognosis for patients with NSCLC is grim (Mulshine J L, et al. 1986, J Clin Oncol; 4: 1704-1715). In a small subset of patients, however, a response to chemotherapy can be observed. In part, these cases might represent NSCLC in which SCLC-components occur since such a heterogeneous composition is quite common in lung cancer (see above).

It may be obvious from these data that alternative treatment modalities for these patients are critical, but a major obstacle to the successful treatment and eradication of lung cancer is its late diagnosis, and shortcoming techniques for a proper classification of the different types of lung cancer.

Lung cancer is often diagnosed by chest radiographs (X-rays), Computed Tomography (CT) imaging, spiral CT, Positron Emission Tomography (PET), scintigraphy, biopsy, biomarker analysis, or sputum cytology. As with any other diagnostic tests, lung cancer diagnostic tests are evaluated using the measures of sensitivity (the proportion of true positives that are correctly identified by the test) and specificity (the proportion of true negatives that are correctly identified by the test). Diagnostic tests often fail due to poor sensitivity and specificity.

Chest X-rays can be capable of diagnosing NSCLC by detecting lesions or cavities formed by squamous cell carcinomas. In general, however, chest X-rays do not detect lung cancers until the cancer has metastasized and complete surgical resection is not possible. CT is used to track the spread of cancer cells, and may be more effective than a standard chest X-ray for the early detection of lung cancer. Spiral CT is a form of CT that may be more sensitive in diagnosing lung cancer at an early stage, however it has been reported to have low specificity and sensitivity with respect to detecting certain types of lung cancer. PET is a sensitive and non-invasive imaging technique that is capable of detecting lung cancers that have spread, for example into the mediastinum, as well as in the lungs. However, the costs associated with PET imaging make it relatively inaccessible for screening purposes. Scintigraphy is an imaging technique in which patients are administered radioactive agents that bind cancer cells. Biopsy involves obtaining lung tissue and cells for diagnosis, and may be performed by thoracoscopy, bronchoscopy (e.g., by bronchoalveolar lavage or BAL), or fine needle procedures.

Biomarkers, such as pRb2/p130, p53, and ras have been implicated as diagnostic agents for lung cancer, but an appropriate set of biomarkers for early diagnosis or to classify the different sets of lung cancers (lung cancer subtyping) is currently lacking. Today, lung cancer is mainly diagnosed using Neuron specific enolase (NSE), the cytokeratin fragment antigen 21.1 (CYFRA 21-1), a cytokeratin of the group (CK4, CK5, CK6, CK7, CK8, CK10, CK13, CK14, CK15, CK16, CK17, CK18, CK19 and CK20), Carcinoambryonic antigen (CEA), Gastrin releasing peptide (GRP-ProGRP), Chromogranin (CHGA), Thyroid transcription factor-1 (TITF-1), synapthophysin (SYPH) and neuroendocrine specific proteins (NSP). In this respect a panel typically used for the neuroendocrine differentiation of lung cancers consist of NSE, SYPH and CHGA. Notwithstanding the value of each of said known lung cancer markers, given the heterogeneity of lung tumor antigens a proper tumor marker panel, with the desired sensitivity and specificity for Non Small Cell Lung Cancer (NSCLC) subtyping as well as for the specific detection of Small Cell Lung Cancer (SCLC) in early disease stages is currently missing. Using the aforementioned tumor markers or tumor marker panels, would result in a high rate of false positives in the diagnosis of SCLC in patients with non-malignant lung disease (e.g. chronic obstructive pulmonary disease (COPD)), patients with Non Small Cell Lung Cancer (NSCLC) and patients with other neuroendocrine (NE) tumors or patients with brain tumors. A further disadvantage of the present markers is that the majority only allows an immunohistochemical staining and not a serological determination.

In clinical practice, accurate diagnosis of various subtypes of cancer is important because treatment options, prognosis, and the likelihood of therapeutic response all vary broadly depending on the diagnosis. Accurate prognosis, or determination of distant metastasis-free survival could allow an oncologist to tailor the administration of adjuvant chemotherapy, with patients having poorer prognoses being given more aggressive treatment. Furthermore, accurate prediction of poor prognosis would greatly impact clinical trials for new lung cancer therapies, because potential study patients could then be stratified according to prognosis. Trials could be limited to patients having poor prognosis, in turn making it easier to discern if an experimental therapy is efficacious. To date, no set of satisfactory predictors for prognosis based on the clinical information alone has been identified.

It would, therefore, be beneficial to provide specific methods and reagents for the (early) diagnosis, subtyping, differentiation, staging, prognosis, monitoring and follow-up of treatment of lung cancer, that overcome the shortcomings of the present diagnostic tools (supra).

Here we show that serological detection of tumor specific antigens by using a combination of specifically selected monoclonal antibodies allows diagnosis and subtyping of lung cancer with high sensitivity and specificity. These tumor specific antigens are released in the peripheral circulation due to necrosis of tumor parts caused by tumor growth beyond the reach of the tumor vascular supply; tumor rejection due to immunological responses; cell death exceeding phagocytic capacity or as a consequence of effective tumor treatment.

### SUMMARY OF THE INVENTION

It is an object of the present invention to provide an in vitro method for (early) diagnosis, subtyping, and determination of the differentiation of lung cancer in a subject, said method comprising the steps of; (a) obtaining a sample from said subject; and (b) determining the expression of at least two tumor marker genes selected from the group consisting of NCAM splice variants NCAM 120, NCAM 140 and/or NCAM 180, a cytokeratin (CK), Neuroendocrine specific protein (NSP)-reticulons (RTN1), Synapthophysin (SYPH), Chromogranin A (CHGA), Thyroid transcription factor-1 (TITF-1), γ Neuron Specific Enolase (γNSE) and Heat Shock Protein-47 (HSP47); wherein the expression of said genes or the presence of said proteins allows to detect and/or diagnose and/or subtype and/or determine the degree of heterogeneity or staging of the lung cancer in said subject.

In a particular embodiment, and as evident from the further embodiments hereinafter, one of the at least two genes in the aforementioned method consists of NCAM 180 of the NCAM splice variant expressing NCAM exon 18.

It is accordingly an objective of the present invention to provide an in vitro method for (early) diagnosis, subtyping, and determination of the differentiation or staging of lung cancer in a subject, said method comprising the steps of; (1) obtaining a sample from said subject; (2) determining the expression of NCAM 180 or the NCAM splice variant expressing the NCAM exon 18; and (3) determine the expression of at least one tumor marker gene selected from the group consisting of NCAM splice variants NCAM 120, or NCAM 140; a cytokeratin (CK); Neuronendocrine specific protein (NSP)-reticulons (RTN1); Synapthophysin (SYPH); Chromogranin A (CHGA); Thyroid transcription factor-1 (TITF-1); γ Neuron Specific Enolase (γNSE) and Heat Shock Protein-47 (HSP47); wherein the expression of said genes or the presence of said proteins allows to detect and/or diagnose and/or subtype and/or determine the degree of heterogeneity or staging of the lung cancer in said subject.

In a particular embodiment of the present invention, the method comprises the steps of; (a) obtaining a sample from said subject; and (b) determining the expression of the tumor marker genes selected from the group consisting of NCAM; the NCAM splice variant expressing NCAM exon 18, a cytokeratin (CK), and Neuroendocrine specific protein (NSP)-reticulons (RTN1); wherein the expression of said genes or the presence or absence of said proteins allows to detect and/or diagnose and/or subtype and/or determine the degree of heterogeneity or staging of the lung cancer in said subject.

The cytokeratin as used herein is typically selected from the group consisting of CK4, CK5, CK6 (being two cytokeratin genes/proteins CK6a and CK6b), CK7, CK8, CK10, CK13, CK14, CK15, CK16, CK17, CK18, CK19 and CK20. In a particular embodiment of the methods according to the invention, the expression of at least two cytokeratins is being determined. In an even further embodiment, the expression/presence of at least 3, 4, 5, 6, 7, 8, 9, 10, 11, 12, 13, or 14 of said CK genes/proteins is used. As exemplified hereinafter, in one embodiment of the invention, the cytokeratins as used herein are selected from CK6 (being two cytokeratin genes/proteins CK6a and CK6b), CK16 and CK17.

In subtyping or staging the different types of lung cancer, the expression of 2 or more of the aforementioned genes is used; in a particular embodiment the expression of at least 3, 4, 5, 6, 7, 8, 9, 10, 11, 12, 13 or 14 of said genes is determined. In an even further embodiment, the expression of all of the aforementioned genes is used in the method of subtyping lung cancer in a subject.

In one embodiment of the method according to the invention, the expression of NCAM 180, and in particular the expression of the NCAM splice variant expressing the NCAM exon 18-antigen, is used in combination with one or more of the tumor markers mentioned in step (3) hereinbefore to differentiate SCLC from NSCLC. In a more particular embodiment, NCAM 180, and in particular the expression of the NCAM splice variant expressing NCAM exon 18, is used in combination with one or more (i.e. 2, 3, 4, 5, 6, 7, 8, 9 or all) of the cytokeratin genes selected from the group consisting of CK4, CK5, CK6, CK10, CK13, CK14, CK15, CK16, CK17 and CK20 to differentiate SCLC from NSCLC.

In said embodiment;

SCLC is characterized by the expression of NCAM 180 (i.e. the NCAM splice variant expressing NCAM exon 18), CK8 and CK18 in the absence of the cytokeratin genes CK4, CK5, CK6, CK10, CK13, CK14, CK15, CK16, CK17 and CK20
and
NSCLC is characterized by the expression of CK19, CK6/CK16/CK17 (i.e. the couple of CK6 and CK16 and CK17) and/or CK8/CK18 (i.e. the couple of CK8 and CK18) in the absence of NCAM 180 (i.e. the NCAM splice variant expressing NCAM exon 18); in particular CK19, CK6/CK16/CK17 or CK8/CK18 in the absence of NCAM 180 (i.e. the NCAM splice variant expressing NCAM exon 18).

In a second embodiment, the expression of NCAM 180 (i.e. the NCAM splice variant expressing NCAM exon 18) is used with one or more (i.e. 2, 3, 4, 5, 6, 7, 8, 9, 10, 11 or all) of the cytokeratin genes CK4, CK5, CK6, CK7, CK8, CK10, CK13, CK14, CK15, CK16, CK17, CK18, CK19 and/or CK20 to detect and/or diagnose NSCLC and/or to determine or subtype NSCLC's (i.e. to subtype adenocarcinoma versus squamous cell carcinoma) in a subject.

In one objective of said second embodiment, adenocarcinoma differentiation or subtype of NSCLC is characterized by the expression of at least CK7, CK8/CK18 (i.e. the couple of CK8 and CK18) and CK19; and the absence of CK20, NCAM180 (i.e. the NCAM splice variant expressing NCAM exon 18) and one or more (i.e. 2, 3, 4, 5, 6, 7, 8, or all) of the cytokeratin genes selected from the group consisting of CK4, CK5, CK6, CK10, CK13, CK14, CK15, CK16 and CK17.

In a further objective of said second embodiment, squamous cell carcinoma (SCC) differentiation or subtype of NSCLC is characterized by the expression of one or more (i.e. 2, 3, 4, 5, 6, 7, 8, 9 or all) of the cytokeratin genes selected from the group consisting of CK4, CK5, CK6, CK10, CK13, CK14, CK15, CK16, CK17 and CK19 in the absence of NCAM 180 CK20, and CK7. In an even further objective of this second embodiment, the squamous cell carcinoma G2 and not G3 stage of the SCC subtype of NSCLC is characterized by the expression of the cytokeratin gene CK17 in the absence of NCAM 180, CK20, and CK7.

In a third embodiment the expression of NSP-reticulon (a.k.a. RTN1), NCAM, NSE, SYPH and/or CHGA is additionally used to determine the neuroendocrine differentiation of lung cancers. In this alternative embodiment neuroendocrine differentiation is characterized by the expression of at least two genes selected from NSE, SYPH and/or CHGA; in particular the expression of NSE, SYPH and/or CHGA; more in particular NSP and/or NSE is (are) used to differentiate lung cancers with and without neuroendocrine differentiation. This sub-classification of lung cancers with and without neuroendocrine origin can be of importance since targeted therapies will become more and more common.

In analogy, the additional expression of at least one gene selected from the group consisting of CK4, CK5, CK6, CK10, CK13, CK14, CK15, CK16, CK17 and CK19, NSE and HSP47 in the absence of CK20 and NCAM 180 can be used to differentiate Squamous from non-Squamous NSCLC, being adenocarcinoma or other NSCLC-subtypes.

In an even further objective of said third embodiment, the expression of CK8, CK18 and CK20 can be used to detect and/or diagnose neuroendocrine Merkel Cell carcinomas.

It is also an objective to provide an in vitro method for (early) diagnosis, subtyping, and determination of the differentiation of lung cancer in a subject, said method comprising the steps of; (a) obtaining a sample from said subject; and (b) determining the expression of the tumor marker genes selected from the group consisting of consisting of NCAM, NCAM exon 18, NSP and two or more cytokeratin antigen (with in particular CK6, CK16 and CK17; wherein the expression of said genes or the presence or absence of said proteins allows to detect and/or diagnose and/or subtype and/or determine the degree of heterogeneity or staging of the lung cancer in said subject.

In any one of the aforementioned methods, the sample is selected from the group consisting of blood, serum, plasma, urine, saliva, semen, breast exudates, cerebrospinal fluid, tears, sputum, mucous, lymph, pleural effusions, tumor tissues and bronchioalveolar lavages.

The expression level of the marker genes mentioned hereinbefore, is determined using art known procedures typically done at the protein or the nucleic acid level.

Methods to determine the expression level include;
an immunoassay, wherein the expression level is determined using antibodies that specifically bind to the proteins encoded by said genes; or
a hybridization assay, wherein the expression level is determined using a probe that hybridizes to the nucleic acid molecules encoding said genes.
Immunohistochemistry wherein the described panel is used for in vitro diagnosis/sub-typing and staging of said tumor antigens in tumor tissues
Imaging wherein the described panel is used for in vivo diagnosis, monitoring of disease progression or treatment response.

For NCAM 180 for example, the expression of the gene or the gene product is specifically detected by antibodies or DNA-probes specific for the NCAM exon 18 region. As provided in the examples hereinafter, specific antibodies for NCAM exon 18 include but are not limited to the monoclonal antibodies MUMI21B1, MUM1, MUM4 and MUM6.

In a further aspect, the present invention provides the use of the genes as identified hereinbefore, in monitoring the response of a subject to lung cancer treatment.

In one embodiment the genes are used in in vivo imaging of lung cancer, more in particular the genes selected from the group consisting of NCAM, NCAM exon 18, CK6/16/CK17, RTN1, SYPH, CHGA and NSE; are used in these in vivo imaging techniques.

Art known in vivo imaging techniques can be used, such as for example using computed tomography (CT), Positron Emission Tomography (PET) and/or PET-CT.

A kit for subtyping or staging lung cancer in a subject, comprising a reagent to determine the expression of a gene as defined in herein is also an aspect of the present invention.

These and other aspects of the invention are described herein in more detail.

### Description of Sequences.

SEQ ID NO:1 is the nucleotide sequence for human NCAM exon 18.

SEQ ID NO:2 is the amino acid sequence for human NCAM exon 18.

SEQ ID NO:3 is the nucleotide sequence for human NCAM 180.

SEQ ID NO:4 is the amino acid sequence for human NCAM 180.

SEQ ID NO:5 is the nucleotide sequence for a fragment of human NCAM exon 18.

SEQ ID NO:6 is the amino acid sequence for the fragment of human NCAM exon 18 encoded by SEQ ID NO:5

### BRIEF DESCRIPTION OF THE DRAWINGS

FIG. 1: Schematic representation of the principle of the differential expression PCR. For primer set A, the forward primer was designed in exon 17, the reverse primer in exon 19. For primer set B, the forward as well as the reverse primer was designed in NCAM exon 18. PCR amplification of cells expressing NCAM 140 resulted in a 180 by PCR product with primerset A, and no amplicon with primerset B. Cells expressing NCAM 180 produce a 600 by PCR amplicon with primerset B. Cells expressing both NCAM 140 and NCAM 180 produce PCR products with both primersets.

FIG. 2: Overexpression of NCAM exon 18 as part of NCAM 180 in cell cultures derived from small cell lung cancers (SCLC, N=4), no expression in non small cell lung cancers (NSCLC, N=5) and peripheral blood mononuclear cells (PBMC) of healthy controls and low to medium expression in cell lines derived from neuroendocrine tumors (SH-SYSY and CCI). Cells expressing NCAM exon 18, result in a 604 by PCR product in the NCAM exon 18 specific PCR. Cells expressing the NCAM 140 kDa splice variant have a 180 by product for the NCAM exon 17-19 PCR amplification reaction.

FIG. 3: NCAM antigen detection in serum of lung cancer patients (N=7, black bars) and controls (N=7, grey bars). Serum antigen levels were measured using a sandwich ELISA. ELISA plates were coated with an NCAM specific monoclonal capture antibody (123C3) and blocked with BSA. 1:4 diluted serum was incubated and a biotin-labelled NCAM specific detection antibody (RNL-1) added. NCAM serum antigen level detection was performed using peroxidase conjugated streptavidin and a TMB substrate. OD₄₅₀ values representing the level of NCAM antigen as measured in the serum samples are shown for patients and controls.

FIG. 4: NSP expression in primary human lung tumors as shown by immunohistochemistry.

FIG. 5: NCAM exon 18-antigen detection in serum of lung cancer patients (N=7, black bars) and controls (N=7, grey bars). Serum NCAM exon 18-antigen levels were measured using a sandwich ELISA. ELISA plates were coated with capture antibody and blocked with BSA. The used capture antibodies were for A-C: MUMI21B2 and for D: RNL-1. 1:4 diluted serum samples were incubated and a biotin-labelled detection antibody added. The used detection antibodies were for A: MUM1, for B: MUM4, for C: MUM6 and for D: MUMI21B2. Antigen detection was performed using peroxidase conjugated streptavidin and a TMB substrate. For NCAM exon 18-antigen detection we used 4 different antibody couples.

FIG. 6: Immunohistochemistry on tissue arrays revealing the expression of proteins found to be overexpressed in squamous cell carcinoma (SCC). A representative picture of bronchial epithelium, squamous cell carcinoma, adenocarcinoma and large cell carcinoma staining is shown for each antibody.

FIG. 7: Diagnostic guideline using the lung cancer subtype specific biomarkers. Positive serum antigen titer: black; No serum antigen titer: white; Disease stage specific reactivity that may differ between antibody couples: Light grey. Adeno: Adenocarcinoma; NE: Neuroendocrine; LC: Large Cell carcinoma; COPD: Chronic Obstructive pulmonary disease, SCLC: Small Cell Lung Carcinoma, NSCLC: Non Small Cell Lung Carcinoma.

### DETAILED DESCRIPTION OF THE INVENTION

The invention provides, biomarkers, e.g., nucleic acid molecules and expression products thereof, that are differentially expressed in healthy cells derived from subjects having a lung cancer and/or in malignant lung cancer cells, compared to healthy cells derived from subjects without cancer. The biomarkers can be used in a rapid multifactorial assay for early detection of lung cancer, to differentiate the different sub types and to determine the degree of heterogeneity of lung cancers known.

### Cancers

By a “cancer” or “neoplasm” is meant any unwanted growth of cells serving no physiological function. In general, a cell of a neoplasm has been released from its normal cell division control, i.e., is a cell whose growth is not regulated by the ordinary biochemical and physical influences in the cellular environment. In most cases, a neoplastic cell proliferates to form a clone of cells that can be malignant.

The term cancer includes cell growths that are technically benign but which carry the risk of becoming malignant. By “malignancy” is meant an abnormal growth of any cell type or tissue. The term malignancy includes cell growths that are pre-malignant.

This term also includes any cancer, carcinoma, neoplasm, neoplasia, or tumor. Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to mestastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body).

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body that is different from the primary site. Cancers named based on primary site may be correlated with histological classifications. For example, lung cancer or bronchogenic carcinoma of the lung generally arises in epithelial cells in the lung, and is generally categorized into “small cell carcinoma” or “SCC” and “non-small cell lung carcinoma” or “NSCLC.” NSCLC includes adenocarcinoma, squamous cell carcinoma, and large cell carcinoma. As already outlined hereinbefore, the biomarkers of the present invention are particularly useful to characterize the different types of lung cancer in a patient.

### Biomarkers

The invention provides biomarkers, e.g., nucleic acid molecules and expression products thereof, that are differentially expressed in histologically normal cells derived from subjects having a lung cancer and/or in malignant lung cancer cells, compared to normal cells derived from subjects without cancer.

A “biomarker” is a molecular indicator of a specific biological property and as used herein is a nucleic acid molecule (e.g., a gene or gene fragment) or an expression product thereof (e.g., a polypeptide or peptide fragment or variant thereof) whose differential expression (presence, absence, over-expression or under-expression relative to a reference) within a cell or tissue indicates the presence or absence of a lung cancer. An “expression product” as used herein is a transcribed sense or antisense RNA molecule (e.g., an mRNA), or a translated polypeptide corresponding to or derived from a polynucleotide sequence. In some embodiments, an expression product can refer to an amplification product (amplicon) or cDNA corresponding to the RNA expression product transcribed from the polynucleotide sequence. A “panel” of biomarkers is a selection of two or more combinations of biomarkers.

By “differential expression” or “differentially expressed” is meant a difference in the frequency or quantity, or both, of a biomarker in a cell or tissue or sample derived from a subject having a lung cancer compared to a reference cell or tissue or sample, e.g., in a malignant lung cancer cell and/or in a normal cell derived from a subject having a lung cancer (i.e., a cell having a malignancy associated change) compared to a reference or normal cell e.g., a cell derived from a subject without cancer or with undetectable cancer or a normal cell derived from a subject who has undergone successful resection of lung cancer. In some embodiments, the control or reference cell may be a SCLC or a NSCLC. In some embodiments, differential expression refers to a difference in the frequency or quantity, or both, of a biomarker in a malignant lung cancer cell compared to the reference cell. For example, differential expression of a biomarker can refer to an elevated level or a decreased level of expression of the biomarker in samples of lung cancer patients compared to samples of reference subjects, e.g. measurement of protein level or antibody titer in blood, urine, saliva, serum, pleural effusions or bronchioalveolar lavages samples taken from lung cancer patients compared to the measurement of protein level or antibody titer in blood, urine, saliva, serum, pleural effusions or bronchioalveolar lavages samples taken from non-lung cancer controls, including healthy subjects and subjects with respiratory airway infections like bronchitis and bronchiolitis. Alternatively or additionally, differential expression of a biomarker can refer to detection at a higher frequency or at a lower frequency of the biomarker in samples of lung cancer patients compared to samples of reference subjects. A biomarker can be differentially present in terms of quantity, frequency or both. In some embodiments, differential expression of the biomarkers of the invention may be measured at different time points, e.g., before and after therapy. By “level of expression” is meant the level of mRNA, as well as pre-mRNA nascent transcript(s), transcript processing intermediates, mature mRNA(s), and degradation products, encoded by a gene in the cell, and/or the level of protein, protein fragments, and degradation products in a cell.

The difference in quantity or frequency or both of a biomarker may be measured by any suitable technique, such as a statistical technique. For example, a biomarker can be differentially expressed between a lung cancer sample and a reference sample, if the frequency of detecting the biomarker in a lung cancer sample is significantly higher or lower than in the reference sample, as measured by standard statistical analyses such as student's t-test, where p<0.05 is generally considered statistically significant. In some embodiments, a biomarker is differentially expressed if it is detected at least about 20, 30, 40, 50, 60, 70, 80, 90, 100% or more or 2-, 5-, 10 or more fold more or less frequently in a lung cancer compared to a reference sample. Alternatively or additionally, a biomarker is differentially expressed if the amount of the biomarker in a lung cancer is statistically significantly different, e.g., by at least 20, 30, 40, 50, 60, 70, 80, 90, 100% or more or 2-, 5-, 10 or more fold when compared to the amount of the biomarker in a reference sample or if it is detectable in one sample and not detectable in the other. In some embodiments, differential expression may refer to an increase or decrease in expression of at least 20, 30, 40, 50, 60, 70, 80, 90, 100% or more or 2-, 5-, 10 or more fold, in a test sample relative to a reference sample.

A “sample” can be any organ, tissue, cell, or cell extract isolated from a subject, such as a sample isolated from a mammal having a lung cancer or at risk for a lung cancer (e.g., based on family history or personal history, such a heavy smoking). For example, a sample can include, without limitation, cells or tissue (e.g., from a biopsy or autopsy) solid lung tumors, sputum, cough, bronchoalveolar lavage, bronchial brushings, buccal mucosa, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, tissue or fine needle biopsy samples, and pleural fluid, etc. isolated from a mammal with a lung cancer, or any other specimen, or any extract thereof, obtained from a patient (human or animal), test subject, healthy volunteer, or experimental animal. A subject can be a human, rat, mouse, non-human primate, etc. A sample may also include sections of tissues such as frozen sections taken for histological purposes. A “sample” may also be a cell or cell line created under experimental conditions, that is not directly isolated from a subject.

A “control” or “reference” includes a sample obtained for use in determining base-line expression or activity. Accordingly, a control sample may be obtained by a number of means including from non-cancerous cells or tissue e.g., from cells surrounding a tumor or cancerous cells of a subject; from subjects not having a cancer; from subjects not suspected of being at risk for a cancer; or from cells or cell lines derived from such subjects. A control also includes a previously established standard, such as a previously characterized SCLC, NSCLC including SQC, AC and NSCLC with or without neuroendocrine origin. Accordingly, any test or assay conducted according to the invention may be compared with the established standard and it may not be necessary to obtain a control sample for comparison each time.

Biomarkers for sybtyping the different kind of lung cancers, according to the invention, include NCAM 120, NCAM 140, NCAM 180, a cytokeratin (CK), neuroendocrine specific protein (NSP)-reticulon 1A (RTN1), Synapthophysin (SYPH), Chromogranin (CHGA), Thyroid transcription factor (TITF-1), neuron specific enolase (NSE) and HSP47. Two or more of these biomarkers, e.g., 2, 3, 4, 5, 6, 7, etc. of the biomarkers, up to all of the biomarkers, may be used together in any combination in an assay according to the invention. In some embodiments, one or more of the biomarkers may be specifically excluded from an assay (supra). In some embodiments, particular combinations will be used, for example in differentiating SCLC and NSCLC. In a particular embodiment of the present invention NCAM 180 is used in combination with at least one or more of the biomarkers selected from the group consisting of NCAM 120, NCAM 140, a cytokeratin (CK), reticulon 1A (RTN1), CD45, Synapthophysin (SYPH), Chromogranin (CHGA), Thyroid transcription factor (TITF-1), γ-neuron specific enolase (γ-NSE) and HSP47. In said embodiment the NCAM 180 kDa splice variant expression is specifically determined by antibodies or probes for the NCAM exon-18 region.

Biomarkers according to the invention include substantially identical homologues and variants of the nucleic acid molecules and expression products thereof described herein, for example, a molecule that includes nucleotide sequences encoding polypeptides functionally equivalent to the biomarkers of the invention, e.g, sequences having one or more nucleotide substitutions, additions, or deletions, such as allelic variants or splice variants or species variants or molecules differing from the nucleic acid molecules and polypeptides referred to in the Tables herein due to the degeneracy of the genetic code. Species variants are nucleic acid sequences that vary from one species to another, although the resulting polypeptides generally will have significant amino acid identity and functional similarity relative to each other. A polymorphic variant (e.g., a single nucleotide polymorphism or SNP) is a variation in the nucleic acid sequence of a particular gene between individuals of a given species.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 10% to 99%, or more generally at least 10%, 20%, 30%, 40%, 50, 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various, deletions, substitutions, and other modifications. Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO4, pH 7.2, 7% SDS, 1 mM EDTA, and 1% BSA (fraction V), at a temperature of 65[deg.]C, or a buffer containing 48% formamide, 4.8×SSC, 0.2 M Tris-Cl, pH 7.6, 1×Denhardt's solution, 10% dextran sulfate, and 0.1% SDS, at a temperature of 42[deg.]C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

### Preparation of Reagents Using Biomarkers

The biomarkers described herein may be used to prepare oligonucleotide probes and antibodies that hybridize to or specifically bind the biomarkers listed in the Tables herein, and homologues and variants thereof.

### Antibodies

An “antibody” includes molecules having antigen-binding regions, such as whole antibodies of any isotype (IgG, IgA, IgM, IgE, etc.) and fragments thereof. Antibody fragments include Fab′, Fab, F(ab′)₂, single domain antibodies, Fv, scFv, etc. Antibodies may be prepared using standard techniques of preparation as, for example, described in Harlow and Lane (Harlow and Lane Antibodies; A Laboratory Manual, Cold Spring Harbor Laboratory, Cold Spring Harbor, N.Y., 1988), or known to those skilled in the art. For example, a coding sequence for a polypeptide biomarker of the invention may be purified to the degree necessary for immunization of rabbits. To attempt to minimize the potential problems of low affinity or specificity of antisera, two or three polypeptide constructs may be generated for each protein, and each construct may be injected into at least two rabbits. Antisera may be raised by injections in a series, preferably including at least three booster injections. Primary immunizations may be carried out with Freund's complete adjuvant and subsequent immunizations with Freund's incomplete adjuvant. Antibody titres may be monitored by Western blot and immunoprecipitation analyses using the purified protein. Immune sera may be affinity purified using CNBr-Sepharose-coupled protein. Antiserum specificity may be determined using a panel of unrelated proteins. Antibody fragments may be prepared recombinantly or by proteolytic cleavage. Peptides corresponding to relatively unique immunogenic regions of a polypeptide biomarker of the invention may be generated and coupled to keyhole limpet hemocyanin (KLH) through an introduced C-terminal lysine. Antiserum to each of these peptides may be affinity purified on peptides conjugated to BSA, and specificity tested in ELISA and Western blots using peptide conjugates and by Western blot and immunoprecipitation.

Monoclonal antibodies, which specifically bind any one of the polypeptide biomarkers of the invention are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981). Alternatively monoclonal antibodies may be prepared using the polypeptides of the invention and a phage display library (Vaughan et al., Nature Biotech 14:309-314, 1996). Once produced, monoclonal antibodies may also be tested for specific recognition by Western blot or immunoprecipitation.

In some embodiments, antibodies may be produced using polypeptide fragments that appear likely to be immunogenic, by criteria such as high frequency of charged residues. Antibodies can be tailored to minimise adverse host immune response by, for example, using chimeric antibodies that contain an antigen binding domain from one species and the Fc portion from another species, or by using antibodies made from hybridomas of the appropriate species. Such as for example with NCAM 180, the antibodies are tailored to be specific for the NCAM exon 18 region, also known as MUM protein or NCAM-MUM (see PCT publication WO 2007-104511)

An antibody “specifically binds” an antigen when it recognizes and binds the antigen, for example, a biomarker as described herein, but does not substantially recognize and bind other molecules in a sample. Such an antibody has, for example, an affinity for the antigen, which is at least 2, 5, 10, 100, 1000 or 10000 times greater than the affinity of the antibody for another reference molecule in a sample. Specific binding to an antibody under such conditions may require an antibody that is selected for its specificity for a particular biomarker. For example, a polyclonal antibody raised to a biomarker from a specific species such as rat, mouse, or human may be selected for only those polyclonal antibodies that are specifically immunoreactive with the biomarker and not with other proteins, except for polymorphic variants and alleles of the biomarker. In some embodiments, a polyclonal antibody raised to a biomarker from a specific species such as rat, mouse, or human may be selected for only those polyclonal antibodies that are specifically immunoreactive with the biomarker from that species and not with other proteins, including polymorphic variants and alleles of the biomarker. Antibodies that specifically bind any of the biomarkers described herein may be employed in an immunoassay by contacting a sample with the antibody and detecting the presence of a complex of the antibody bound to the biomarker in the sample. The antibodies used in an immunoassay may be produced as described herein or known in the art, or may be commercially available from suppliers, such as Dako Canada, Inc., Mississauga, ON. The antibody may be fixed to a solid substrate (e.g., nylon, glass, ceramic, plastic, etc.) before being contacted with the sample, to facilitate subsequent assay procedures. The antibody-biomarker complex may be visualized or detected using a variety of standard procedures, such as detection of radioactivity, fluorescence, luminescence, chemiluminescence, absorbance, or by microscopy, imaging, etc. Immunoassays include immunohistochemistry, enzyme-linked immunosorbent assay (ELISA), western blotting, immunoradiometric assay (IRMA), lateral flow, evanescence (DiaMed AG, Cressier sur Morat, Switzerland, as described in European Patent Publications EP1371967, EP1079226 and EP1204856), immunohisto/cyto-chemistry and other methods known to those of skill in the art. Immunoassays can be used to determine presence or absence of a biomarker in a sample as well as the amount of a biomarker in a sample. The amount of an antibody-biomarker complex can be determined by comparison to a reference or standard, such as a polypeptide known to be present in the sample. The amount of an antibody-biomarker complex can also be determined by comparison to a reference or standard, such as the amount of the biomarker in a reference or control sample. Accordingly, the amount of a biomarker in a sample need not be quantified in absolute terms, but may be measured in relative terms with respect to a reference or control.

### Probes and Primers

A “probe” or “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are known to those skilled in the art. Probes or primers specific for the nucleic acid biomarkers described herein, or portions thereof, may vary in length by any integer from at least 8 nucleotides to over 500 nucleotides, including any value in between, depending on the purpose for which, and conditions under which, the probe or primer is used. For example, a probe or primer may be 8, 10, 15, 20, or 25 nucleotides in length, or may be at least 30, 40, 50, or 60 nucleotides in length, or may be over 100, 200, 500, or 1000 nucleotides in length. Probes or primers specific for the nucleic acid biomarkers described herein may have greater than 20-30% sequence identity, or at least 55-75% sequence identity, or at least 75-85% sequence identity, or at least 85-99% sequence identity, or 100% sequence identity to the nucleic acid biomarkers described herein. Probes or primers may be derived from genomic DNA or cDNA, for example, by amplification, or from cloned DNA segments, and may contain either genomic DNA or cDNA sequences representing all or a portion of a single gene from a single individual. A probe may have a unique sequence (e.g., 100% identity to a nucleic acid biomarker) and/or have a known sequence. Probes or primers may be chemically synthesized. A probe or primer may hybridize to a nucleic acid biomarker under high stringency conditions as described herein.

Probes or primers can be detectably-labeled, either radioactively or non-radioactively, by methods that are known to those skilled in the art. Probes or primers can be used for lung cancer detection methods involving nucleic acid hybridization, such as nucleic acid sequencing, nucleic acid amplification by the polymerase chain reaction (e.g., RT-PCR), single stranded conformational polymorphism (SSCP) analysis, restriction fragment polymorphism (RFLP) analysis, Southern hybridization, northern hybridization, in situ hybridization, electrophoretic mobility shift assay (EMSA), fluorescent in situ hybridization (FISH), and other methods that are known to those skilled in the art.

By “detectably labeled” is meant any means for marking and identifying the presence of a molecule, e.g., an oligonucleotide probe or primer, a gene or fragment thereof, or a cDNA molecule. Methods for detectably-labeling a molecule are well known in the art and include, without limitation, radioactive labeling (e.g., with an isotope such as ³²P or ³⁵S) and nonradioactive labeling such as, enzymatic labeling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labeled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

### Arrays and Kits

Antibodies, probes, primers and other reagents prepared using the biomarkers of the invention may be used to prepare arrays for use in detecting lung cancer. By “array” or “matrix” is meant refer to a pattern or arrangement of addressable locations or “addresses,” each representing an independent site, on a surface. Arrays generally require a solid support (for example, nylon, glass, ceramic, plastic, etc.) to which the nucleic acid molecules, polypeptides, antibodies, tissue etc. are attached in a specified dimensional arrangement, such that the pattern of hybridization to a probe is easily determinable.

Generally, a probe (e.g., an antibody, nucleic acid probe or primer, polypeptide, etc.) is immobilized on an array surface and contacted with a sample containing a target binding partner (i.e., in the case of an antibody, a polypeptide that specifically binds the antibody, or in the case of a probe, a nucleic acid molecule that hybridizes to the probe) under conditions suitable for binding. If desired, unbound material in the sample may be removed. The bound target is detected and the binding results are analyzed using appropriate statistical or other methods. The probe or the target may be detectably labeled for ease of detection and subsequent analysis. Multiple probes corresponding to the biomarkers described herein may be used. The multiple probes may correspond to one or more of the biomarkers described herein. In addition to probes capable of binding the biomarkers described herein, the arrays may control and reference nucleic acid molecules, polypeptides, or antibodies, to allow for normalization of results from one experiment to another and the comparison of multiple experiments on a quantitative level. Accordingly, the invention provides biological assays using nucleic acid, polypeptide, antibody, or cytology arrays.

The invention also provides kits for detecting lung cancer. The kits may include one or more reagents corresponding to the biomarkers described herein, e.g., antibodies that specifically bind the biomarkers secreted as antigens in the body fluids, recombinant proteins that bind biomarker specific antibodies, nucleic acid probes or primers that hybridize to the biomarkers, etc. In some embodiments, the kits may include a plurality of reagents, e.g., on an array, corresponding to the biomarkers described herein. The kits may include detection reagents, e.g., reagents that are detectably labeled. The kits may include written instructions for use of the kit in (early) detection and subtyping of lung cancer, and may include other reagents and information such as control or reference standards, wash solutions, analysis software, etc.

### Diagnostic and Other Methods

Lung cancers may be diagnosed by detecting the differential expression of one or more of the biomarkers described herein, by immunoassay, such as immunohistochemistry, ELISA, western blotting, or any other method known to those of skill in the diagnostic arts. The detecting may be carried out in vitro or in vivo.

While individual biomarkers are useful diagnostics, the combination of biomarkers as proposed herein, enables accurate (early) diagnosis and subtypes of lung cancer.

Variation in differential expression across multiple biomarkers in different samples can diagnose or predict the presence or absence of a particular type of lung cancer, the response to a particular therapy for lung cancer, or better assess the risk for developing a lung cancer. For example, the expression of NCAM 180 and/or CK8 and CK18 in the absence of CK4, CK5, CK6, CK10, CK13, CK14, CK15, CK16, CK17 and CK20 can be used to detect the presence of a SCLC in a sample. NSCLC is characterized by the absence of NCAM 180 in the presence of CK19, CK6/CK16/CK17 and/or CK8/CK18. Suitable statistical methods and algorithms, e.g., logistical regression algorithm, may be used to analyze and use multiple biomarkers for diagnostic, prognostic, theranostic, or other purposes. The biomarkers (or specific combination of the biomarkers) can be detected and measured multiple times, for example, before, during and after a therapy for lung cancer.

Detection of the biomarkers described herein may be performed as an initial screen for the (early) detection and subtyping of lung cancer and/or may be used in conjunction with conventional methods of lung cancer diagnosis, such as sputum cytology, chest X-ray, CT scans, spiral CT, PET, PET-CT with specific tracers e.g. ⁸⁹Zr, ¹¹C, fluorescent dyes, scintigraphy, biopsy, traditional morphological MACs analysis, etc. Detection of the biomarkers described herein may also be performed in conjunction with previously recognized biomarkers for lung cancer, such as pRb2/p130, p53, and/or ras. Detection of the biomarkers described herein may be performed as part of a routine examination, for example, of heavy smokers over a certain age (e.g., over 60), or may be performed to determine baseline levels of the biomarkers in subjects at risk for lung cancer (e.g., heavy smokers).

In general, the biomarker panel of the present invention, is to be used for molecular imaging (including the aforementioned in vivo imaging techniques, for molecular diagnosis and/or detection and/or to monitor treatment for lung cancer.

Detection of the biomarkers described herein may enable a medical practitioner to determine the appropriate course of action for a subject (e.g, further testing, surgery, no action, etc.) based on the diagnosis. Detection of the biomarkers described herein may also help determine the presence or absence of lung cancer, early diagnosis of lung cancer, prognosis for lung cancer, subtyping of lung cancer, evaluation of the efficacy of a therapy for lung cancer, monitoring a lung cancer therapy in a subject, or detecting relapse of lung cancer in a subject who has undergone therapy for lung cancer and is in remission. In alternative aspects, the biomarkers and reagents prepared using the biomarkers may be used to identify lung cancer therapeutics. The kits and arrays can be used to measure biomarkers according to the invention, to diagnose and sub type a lung cancer. The kits can also be used to monitor a subject's response to a lung cancer therapy, enabling the medical practitioner to modify the treatment based upon the results of the test. The kits can also be used to identify and validate lung cancer therapeutics, such as small molecules, peptides, etc.
This invention will be better understood by reference to the Experimental Details that follow, but those skilled in the art will readily appreciate that these are only illustrative of the invention and should not be construed as limiting the scope of the invention. Additionally, throughout this application, various publications are cited. The disclosure of these publications is hereby incorporated by reference into this application to describe more fully the state of the art to which this invention pertains.

### EXAMPLES

The following examples illustrate the invention. Other embodiments will occur to the person skilled in the art in light of these examples.

### Example 1

### Experiments to Investigate the Differential Expression of NCAM 180 (NCAM Exon 18) in Various Cell Lineages

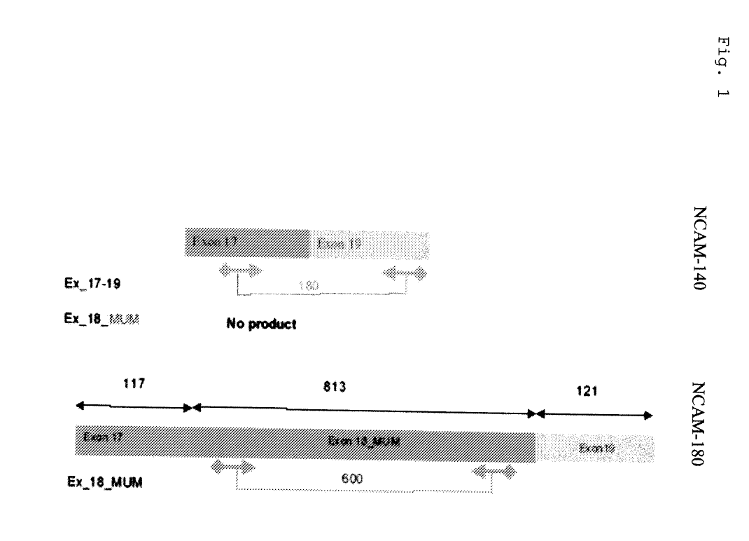

Differential expression of NCAM-180 was evaluated in different cancer cell lines and healthy controls, using art known procedures including;
RNA extraction and cDNA synthesis according to standard procedures; and
PCR amplification to evaluate the expression of NCAM Exon 18 according to the principle represented in FIG. 1.

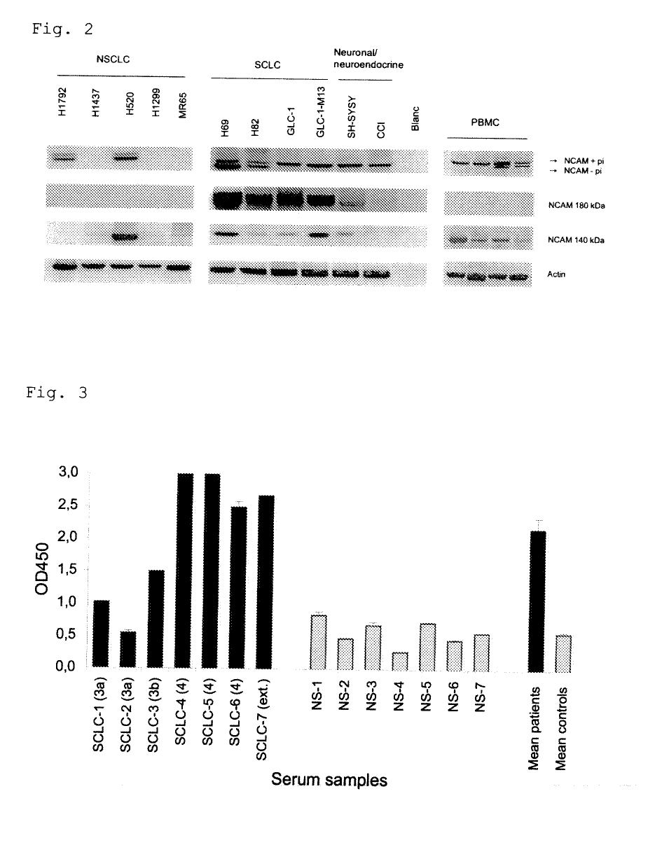

An expression of NCAM Exon 18 as part of NCAM-180 was found in cell cultures derived from neuroendocrine tumors (SH-SYSY and CCI) and a clear over-expression more particularly in Small Cell Lung Cancer (SCLC) cell lines (FIG. 2). No expression of the NCAM 180 kDa splice variant was found in peripheral blood mononuclear cells (PBMC) of healthy controls. The results for the other cell lines are summarized in table 1

TABLE 1
Differential expression of NCAM exon 18 (NCAM 180), and NCAM + pi
or NCAM − pi in cancer cell lines and healthy controls.

| | | NCAM17-19 | NCAM18MUM | NCAM − pi | NCAM + pi | |
|---|---|---|---|---|---|---|
| Cell line | Cancer type | 180 bp | 604 bp | 213 bp | 243 bp | |
| HS-10A | Small cell lung cancer (carcinoma) | ++ | ++ | ++ | + | Lung |
| H69 | Small cell lung cancer (Classic) | ++ | ++ | ++ | + | cancers |
| H82 | Small cell lung cancer (variant) | + | ++ | + | + | |
| GLC-1 | Small cell lung cancer (variant) | + | ++ | − | ++ | |
| GLC-1 M13 | Small cell lung cancer (Classic) | + | ++ | − | ++ | |
| H1437 | Non small cell lung cancer | − | − | − | − | |
| | adenocarcinoma | | | | | |
| H520 | Non-small cell lung cancer | + | − | + | + | |
| H1299 | Non-small cell lung cancer | − | − | − | − | |
| | (from lymph node metastasis) | | | | | |
| H727 | Non-small cell lung cancer | +/− | − | − | + | |
| MR65 | Non small cell lung carcinoma | − | − | − | − | |
| A549 | Non small cell | − | − | − | − | |
| | bronchoepithelial carcinoma | | | | | |
| H1792 | Lung adenocarcinoma (from | − | − | − | − | |
| | metastatic site pleural effusion) | | | | | |
| H460 | Large cell lung cancer (from | ++ | + | ++ | ++ | |
| | metastatic site: pleural effusion) | | | | | |
| H720 | Atypical lung carcinoid | + | − | ++ | ++ | |
| PC3 | Prostate adenocarcinoma (from | − | − | − | − | Non lung |
| | bone metastatis) | | | | | cancers |
| MDA-MB-435s | human breast carcinoma (from | − | − | − | − | |
| | metastatic site: pleural effusion) | | | | | |
| JAR | Placenta Choriocarcinoma | ++ | + | + | − | |
| | (often metastatic) | | | | | |
| A375 | Skin malignant melanoma | ++ | − | ++ | + | |
| A431 | Skin squamous carcinoma | − | − | − | − | |
| SUM 159PT | Anaplastic carcinoma | − | − | − | − | |
| HT29 | Epithelial colon adenocarcinoma | − | − | − | − | |
| Colo205 | Colon adenocarcinoma | − | − | − | − | |
| HCT-116 | Coloncarcinoma | − | − | − | − | |
| MCF7 | Breast adenocarcinoma | − | − | − | − | |
| MCF7-10A | Nontumorigenic breast epithelial | − | − | − | − | |
| | cells (very low ER) | | | | | |
| MDA-MB-239 | Breast carcinoma | − | − | − | − | |
| SUM 149PT | Breast cancer intraductal carcinoma | − | − | − | − | |
| A2780 | Human ovary carcinoma | − | − | − | − | |
| Hela | Cervix cancer | − | − | − | − | |
| LnCap | Human prostate carcinoma | − | − | − | − | |
| DU145 | Human prostate cancer | − | − | − | − | |
| SJSA | Osteosarcoma | ++ | + | ++ | − | |
| HL60 | Promyelocytic leukemia | − | − | − | − | Leukemia |
| Jurkat | T cell lymphoma | − | − | − | − | |
| Molt-4 | Human acute lymphoblastic leukemia | − | − | − | − | |
| K562 | lymphoblast chronic myeloid leukemia | − | − | − | − | |
| SH-SYSY | Human neuroblastoma | ++ | + | − | ++ | Neuronal/ |
| CCI | Astrocytoma | + | +/− | − | ++ | neuro- |
| U87MG | Brain glioblastoma-astrocytoma | + | − | + | + | endocrine |
| CM | Insulinoom | − | − | + | − | |
| Bon-1 | human endocrine pancreatic | +/− | − | − | + | |
| | tumor cell line | | | | | |
| QGP | human pancreatic islet culture | + | − | + | ++ | |
| PBMC_1 | Peripheral blood mononuclear | + | − | + | + | Peripheral |
| | cells of healthy control | | | | | blood |
| PBMC_2 | Peripheral blood mononuclear | + | − | + | + | mono- |
| | cells of healthy control | | | | | nuclear |
| PBMC_3 | Peripheral blood mononuclear | + | − | + | + | cells |
| | cells of healthy control | | | | | |
| PBMC_4 | Peripheral blood mononuclear | + | − | − | + | |
| | cells of healthy control | | | | | |
| PBMC_5 | Peripheral blood mononuclear | + | − | + | − | |
| | cells of healthy control | | | | | |
| PBMC_6 | Peripheral blood mononuclear | + | − | + | + | |
| | cells of healthy control | | | | | |
| PBMC_7 | Peripheral blood mononuclear | + | − | − | ++ | |
| | cells of healthy control | | | | | |
| PBMC_8 | Peripheral blood mononuclear | + | − | − | + | |
| | cells of healthy control | | | | | |
| PBMC_9 | Peripheral blood mononuclear | + | − | + | + | |
| | cells of healthy control | | | | | |
| PBMC_10 | Peripheral blood mononuclear | + | − | + | + | |
| | cells of healthy control | | | | | |
| NHK-10 | normal human keratinocytes | − | − | +/− | + | |
| CH-ME-3 | human foetal microglial cell | − | − | − | − | |

Code: high expression (++), normal expression (+), low expression (+/−), No expression (−).

### Example 2

### Serum Markers for Neuroendocrine Differentiation of Lung Tumors

a. Detection of NCAM Antigen in Human Serum Samples.

NCAM, comprising the splice variants NCAM 120, 140 and 180 is a neuroendocrine differentiation marker. NCAM is expressed in all Small Cell lung carcinoma's (SCLC's) and in 20% of the Non small Cell Lung carcinoma's (NSCLC). Furthermore, NCAM expression is described for all tumors with neuroendocrine differentiation characteristics, for Natural Killer (NK) cells covering 10% of the total Peripheral Blood Mononuclear Cell (PBMC) population and in the stroma of NSCLC. In normal lung tissue on the other hand, NCAM expression is only sporadically found. Here we show that NCAM antigen can be measured in serum of patients (SCLC sera (N=7, PromedDx)) representing tumors with a neuroendocrine differentiation, whereas no NCAM antigens were found in the serum of healthy controls (N=7, healthy volunteers). We used a sandwich ELISA to measure the level of NCAM as a neuroendocrine tumor marker in the serum of patients and controls. Antigen capture was performed using the NCAM specific monoclonal antibody 123C3 (10 ug/ml) and detection was done using the biotinylated NCAM specific monoclonal antibody RNL-1 (20 ug/ml). Both, RNL-1 and 123C3 recognize an epitope in the extracellular region of the NCAM-protein, as such the NCAM 120 as well as the NCAM 140 and NCAM 180 kDa splice variants will be detected. Serum levels of NCAM are measured using a sandwich ELISA. Therefore, NUNC maxisorb 96-microwell plates were coated overnight at 4° C. with an NCAM specific monoclonal capture antibody (10 ug/ml in carbonatebuffer pH 9.5). Plates were washed 2 times with PBST (PBS+0.05% Tween-20), and blocked for 2 h at 37° C. with 4% BSA in PBST. Diluted (in 4% BSA/PBST) serum samples were incubated for 2 h at 37° C., plates were washed 3 times and the biotinylated NCAM antigen specific monoclonal detection antibody (RNL-1-Biotin: 20 ug/ml in PBST+1% BSA) was added. Plates were washed 6 times and Streptavidin-Horse Radish Peroxidase (DAKO P0397) conjugate (1/1000 diluted in PBST+1% BSA) added. Conjugate was incubated for 45 minutes at 37° C. Plates washed 6 times, 3,3′,5,5′-tetramethylbenzidine (TMB) (Calbiochem, CL07) substrate added and the reaction stopped after 15′ at 37° C. using 0.5M H₂SO₄.

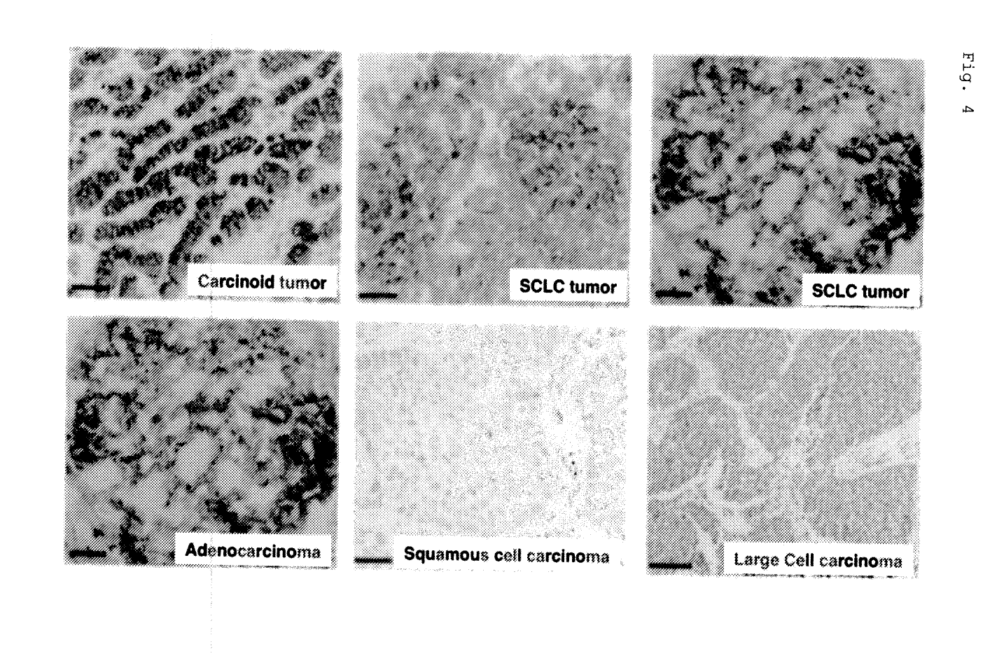

FIG. 3 shows that, using the monoclonal antibody 123C3 as a capture antibody and RNL-1 as a detection antibody all but one SCLC patient sample showed a clear NCAM antigen titer, which was significantly higher in the patient sera as compared to healthy control sera. Here we used serum samples 1:4 diluted. Using undiluted serum samples we expect that the sensitivity of the assay will be 100%, meaning that based on the serum detection of NCAM antigen levels all lung tumors with neuroendocrine characteristics can be diagnosed in serum measuring NCAM antigen titers. These data show that 2 NCAM specific monoclonal antibodies with different epitope specificity can be used to measure NCAM antigen titers in human serum in a simple, high throughput laboratory test (Table 2). Furthermore, these data show that NCAM antigen titers are significantly higher in SCLC patient group as compared to healthy control group (FIG. 3: T-test, p=0.015). These data suggest that serum levels of NCAM antigen can be used as a diagnostic biomarker for neuroendocrine tumors. Together with NSE, SYN, CHGA and SYPH, expression of NCAM can additionally be used as a marker to differentiate lung cancers with and without neuroendocrine origin.

TABLE 2
Biomarker panel for neuroendocrine differentiation of lung tumors

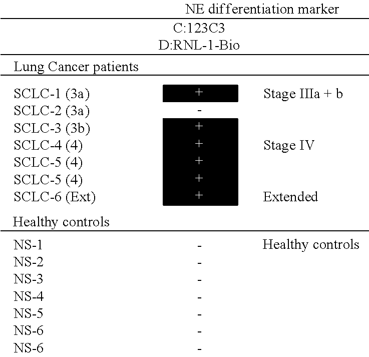

NE: Neuroendocrine;
C: Capture antibody;
D: Detection antibody;
Ext: Extended disease state;
3a, 3b, 4: grade 3a, grade 3b and grade 4 disease stage resp;
Black: Positive serum antigen titer;
White: No serum antigen titer

b. Reticulon Detection as a Marker for Neuroendocrine Differentiation

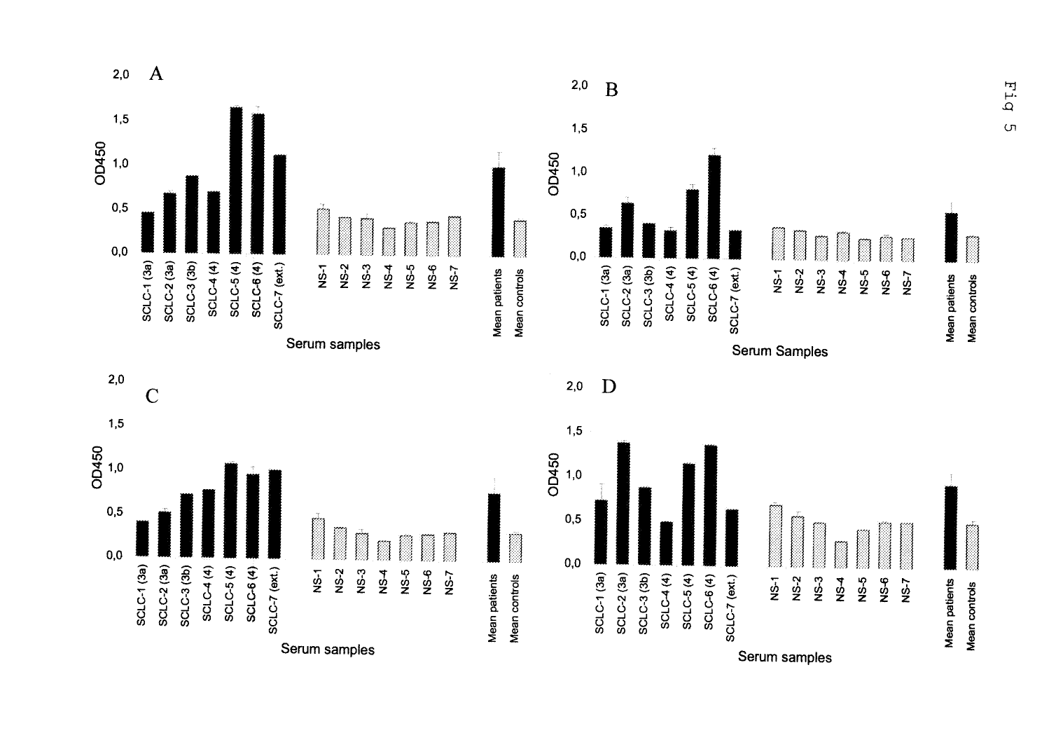

Neuroendocrine-specific proteins (NSPs), also designated reticulon1 (Rtn1), are endoplasmic reticulum associated protein complexes described as markers for neuroendocrine differentiation in normal and malignant cells. NSPs can be expressed as a 135 kDa variant (NSP-A/Rtn-1A), a 45 kDa variant (NSP-B/Rtn-1B) and a 23 kDa variant (NSP-C/Rtn-1C) ¹. Earlier research has shown that NSP expression is a potential biomarker for diagnosis and subtyping of lung tumors with and without neuroendocrine differentiation ². The expression of NSP was studied in normal human and rat tissues, primary human lung tumors e.g. carcinoids, atypical carcinoids, small cell lung carcinoma (SCLC), squamous cell carcinoma (SCC) and adenocarcinoma lung cancer cell lines by immunohistochemistry and Western blot analysis using NSP specific monoclonal antibodies ^{3,4}. In normal human and rat tissues, NSP-A expression was found in neural and neuroendocrine tissues and was shown to be a clear marker for these cells and tissues ³. Next to normal tissues, NSP gene expression was also studied in lung cancer cell lines. Northern blotting shows NSP gene expression in 17/18 SCLC cell lines tested, 14 of these were NSP-A positive. For the non-small cell lung carcinoma (NSCLC) cell lines on the other hand no NSP-A expression was found (0/11) ³. Next, NSP gene expression was studied in primary human tumors (FIG. 4). Using immunohistochemical staining on frozen sections, NSP-A expression was shown in all lung carcinoids (8/8), and in 14/20 SCLC tumor tissues For For the NSCLC tumor tissues studied no NSP gene expression was found, except for NSCLC tissues with neuroendocrine characteristics (NSCLC-NE). These neuroendocrine characteristics were evidenced by the expression of a variety of classical neuroendocrine markers such as neuron specific enolase, chromogranin A, synaptophysin and/or neural cell adhesion molecule (NCAM) ⁴. For 13/27 NSCLC-NE tissues a clear NSP-A expression was shown. These data clearly show that NSP-reticulon expression is restricted to lung carcinoma cells with a neuroendocrine phenotype ³. A neuroendocrine phenotype is very characteristic for all carcinoid tumors of the lung as well as SCLC, but is evident only for approximately 10% of the NSCLC cases. NSCLC with a neuroendocrine phenotype potentially causes differences in treatment response in lung cancer patients as compared to NSCLC without NE characteristics ⁵. One of these differences is the response to chemotherapy, suggesting that neuroendocrine subtyping of NSCLCs is of major importance for the determination of the best treatment protocol.

These data suggest that NSP expression can be used as a biomarker for neuroendocrine differentiation and differential diagnosis of lung tumors.

We expect that NSP protein can be used as a serum biomarker for neuroendocrine differentiation of lung cancer using the reticulon specific RNL-2 and RNL-3 monoclonal antibodies in an appropriate diagnostic detection assay. We suggest that the use of NSP specific monoclonal antibodies combined with the classical neuroendocrine differentiation markers (NCAM, SYN, NSE and CHGA) will result in a highly sensitive diagnosis of tumors with neuroendocrine characteristics. To our knowledge it will be the first report on measuring serum levels of reticulons for the diagnosis of lung cancers with neuroendocrine characteristics.

### Example 3

### Experiments to Measure Serum Levels of NCAM 180/NCAM Exon 18-Antigen

NCAM exon 18 is specifically expressed in the NCAM 180 kDa splice variant of the NCAM protein. NCAM exon 18 is specifically expressed in the cytoplasmic tail of the transmembrane glycoprotein NCAM. This NCAM splice variant is SCLC specific (PCT publication WO 2007-104511). Here we show that NCAM exon 18-antigen can be measured in serum of SCLC patients and that the NCAM Exon 18-antigen titer is significantly higher in serum of SCLC patients (N=7) as compared to healthy controls (N=7). These data suggest that NCAM exon 18 serum antigen titers can be used as a biomarker for SCLC diagnosis. The levels of the NCAM exon 18-tumor antigen are measured in the serum samples using a sandwich ELISA. SCLC serum samples (N=7, stage 3a (N=2), 3b (N=1), 4 (N=3) and extended disease stage (N=1)) were obtained from PromedDx, control sera were isolated from clotted blood obtained from healthy volunteers (smokers and non smokers). Serum levels of NCAM exon 18-antigen were measured using a sandwich-ELISA. For this assay, NUNC maxisorb 96-microwell plates were coated overnight at 4° C. with an NCAM exon 18-antigen specific monoclonal capture antibody (10 ug/ml in carbonatebuffer pH 9.5). Plates were washed 2 times with PBST (PBS+0.05% Tween-20), and blocked for 2 h at 37° C. with 4% BSA in PBST. Diluted (in 4% BSA in PBST) serum samples were incubated for 2 h at 37° C., plates were washed 3 times and the biotinylated NCAM exon 18-antigen specific monoclonal detection antibody (MUM1, MUM4 or MUMI21B2: 20 ug/ml; MUM6: 80 ug/ml in PBST+1% BSA) was added. Plates were washed 6 times and Streptavidin-Horse Radish Peroxidase (DAK0, P0397) conjugate (1/1000 diluted in PBST+1% BSA) added. Conjugate was incubated for 45 minutes at 37° C. Plates washed 6 times, 3,3′,5,5′-tetramethylbenzidine (TMB) (Calbiochem, CL07) substrate added and the reaction stopped after 15′ at 37° C. using 0.5M H₂SO₄. We used different capture-detection antibody couples for the detection of NCAM exon 18-antigen serum levels.

| | | Concentration of |
|---|---|---|
| Capture antibody | Detection antibody | detection antibody |
| (10 ug/ml) | (Biotinylated) | (ug/ml) |
| MUMI21B2 | MUM1 | 20 |
| MUMI21B2 | MUM4 | 20 |
| MUMI21B2 | MUM6 | 80 |
| RNL-1 | MUMI21B2 | 20 |

Results of the detection of NCAM exon 18-antigen serum levels in SCLC sera (N=7) and controls (N=7) are shown in FIG. 5. Detection of NCAM exon 18-antigen in serum was performed using a sandwich ELISA with 4 different capture/detection monoclonal antibody couples. The MUMI21B2, MUM1, MUM4 and MUM6 monoclonal antibodies used for serum diagnosis are NCAM exon 18-antigen specific antibodies, they all recognize a different epitope of the NCAM exon 18-antigen. Our data show that using MUMI21B2 as a capture antibody and MUM1 as a detection antibody for 3/7 (43%) SCLC patient sera the NCAM exon 18-antigen titer was clearly higher as compared to the titer in healthy control sera. Using this couple of capture-detection antibodies the mean NCAM exon 18-antigen expression in the SCLC patient group (N=7) is not significantly higher as compared to the expression in healthy control group (N=7). Using MUMI21B2 as a capture antibody and MUM4 as a detection antibody we found a clear NCAM exon 18-antigen titer in the sera of 4/7 (57%) SCLC patients whereas using MUMI21B2 as a capture and MUM6 as a detection antibody an increased NCAM exon 18-antigen titer was found for 6/7 (86%) of the SCLC patients. For these, the mean NCAM exon 18-antigen titer is significantly higher in the SCLC patient group as compared to the healthy control group (T-Test, p=0.015 for MUMI21B2-MUM4 and T-test, p=0.018 for MUMI21B2-MUM6). In summary NCAM exon 18-antigen detection can be done using the NCAM exon 18-antigen specific monoclonal antibody MUMI21B2 as a capture antibody. We also performed a detection of NCAM exon 18-antigen titers using RNL-1 as capture antibody. RNL-1 is an NCAM specific monoclonal antibody recognizing an epitope in the extracellular region of the transmembrane glycoprotein. Hereby, 6/7 (86%) of the SCLC sera showed a clear NCAM exon 18-antigen titer as compared to 7 controls. Only for SCLC-1 serum the NCAM exon 18-antigen titer measured was not significantly higher as in the healthy controls. For this patient none of the capture detection antibodies gave an NCAM exon 18-antigen titer clearly different from the titer in healthy controls. Although these detections are performed on 1:4 diluted serum, which significantly lowers the detection limit. Hence it is likely that with the use of undiluted serum in the sandwich ELISA higher NCAM exon 18-antigen titers will be obtained. Overall, the mean level of NCAM exon 18-antigen expression was significantly higher in the SCLC patient group as compared to the controls (p=0.018) using RNL-1 as the capture antibody and MUMI21B2 as the detection antibody.

Our data indicate that detection of NCAM exon 18-antigen in patient sera can be used as a potential biomarker to differentiate SCLC patients from healthy controls (Table 3). To our knowledge this is the first time NCAM exon 18-antigen levels were measured in serum of patients and controls and used for diagnosis of SCLC. Our data suggest that the use of NCAM 180-antigen specific monoclonal antibody panels in combination with the expression of CK8 and CK18 and the absence of CK4, CK5, CK6, CK10, CK13, CK14, CK15, CK16, CK17 and CK20, can improve the sensitivity of SCLC diagnosis. By using the combination of markers SCLC patients will be clearly discriminated from healthy controls, NSCLC patients and patients with chronic obstructive pulmonary diseases (COPD). When no expression is found for NCAM 180 in the presence of CK19, CK6/CK16/CK17 and/or CK8/CK18 the diagnosis will be NSCLC.

TABLE 3
Biomarker panels for SCLC diagnosis

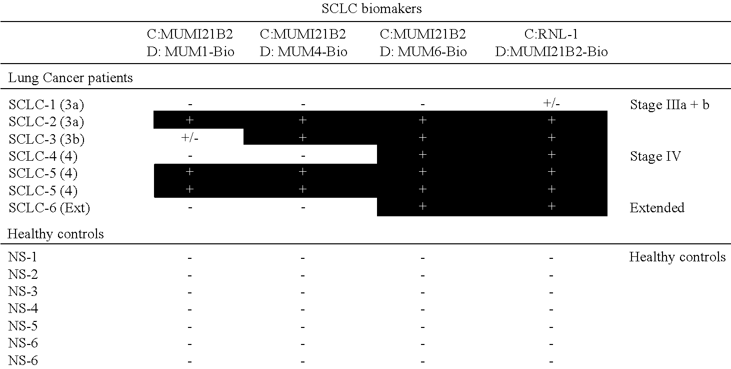

SCLC: Small Cell Lung Cancer;
C: Capture antibody;
D: Detection antibody;
Ext: Extended disease state;
3a, 3b, 4: grade 3a, grade 3b and grade 4 disease stage resp.;
Black: Positive serum antigen titer;
Grey: Medium/low serum antigen titer,
White: No serum antigen titer

### Example 4

### Specific Cytokeratins (CK6/CK16 and CK17) as Biomarkers for Squamous Cell Carcinoma Differentiation and Staging

We used a proteomics approach to identify potential lung cancer biomarkers for squamous cell carcinoma (SCC) ⁶. Because of the heterogeneity of cell types in lung cancer tissue we used microdissection to isolate cells from histologically defined areas to obtain as homogeneous tumor cell material as possible. Proteome analysis could be performed on the limited amount of sample material as obtained by microdissection using a method combining two-dimensional polyacrylamide gel electrophoresis (2D-PAGE) and high sensitive labeling of proteins with fluorescence cyanine dyes (Cy3 and Cy5) via reduced thiol groups of cysteines ⁷. Proteome analysis was performed on microdissected tissue material from normal human bronchial epithelium (N=7) and squamous cell carcinoma tumors of histopathological grade G2 (N=7) and G3 (N=7).

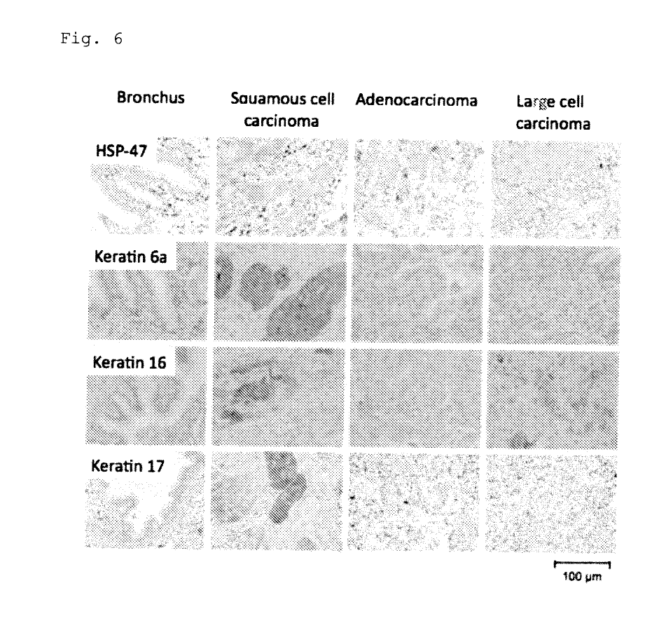

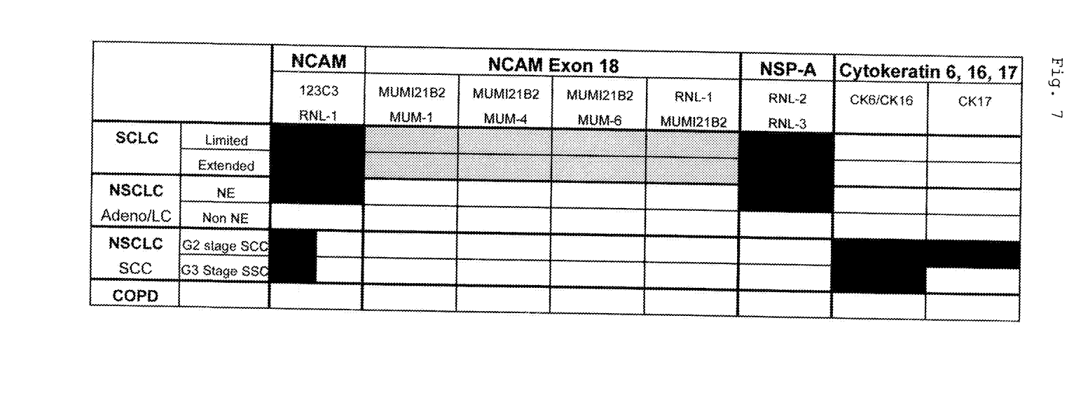

Proteome hits were validated by immunohistochemistry on tissue arrays made of representative areas of squamous cell carcinoma (N=15), adenocarcinoma (N=9), large cell carcinoma (N=5) and normal bronchial epithelium (N=23) to validate the expression of the proteins identified. Proteome analysis of microdissected material resulted in 2500 protein spot of which 85 were significantly differentially expressed between bronchial epithelium on the one hand and G2 or G3 grade tumors on the other hand. Most of the protein spots (88%) were higher abundant in the tumor tissues as compared to the bronchial epithelium. Using MALDI-MS and nano-HPLC/ESI-MS/MS analysis of the generated peptides by tryptic in gel digestion, the identity of 46 protein spots was determined. Most of the identified proteins are involved in protein metabolism (25%), metabolism and energy pathways (31%) cell growth and maintenance (28%), being pathways potentially altered in cancer cells. On basis of their potential implication in tumor biology, we selected HSP-47, cytokeratin 6, cytokeratin 16 and cytokeratin 17 as proteins for further validation. Therefore, we studied the expression on cellular level by immunohistochemistry on tissue arrays (FIG. 6). The immunhistochemical analysis showed a high expression of cytokeratin 6a (CK6a), cytokeratin 16 (CK16) and cytokeratin 17 (CK17) in SCC tumor tissue as compared to normal bronchial epithelium. Both CK6a and CK16 are significantly overexpressed in hyperproliferative squamous cell epithelium, and therefore also in squamous cell carcinoma. No expression is found in normal bronchus, adenocarcinoma or large cell carcinoma. CK 17 is highly expressed in squamous cell carcinoma, no expression was found in adenocarcinoma or large cell carcinoma. A clear expression was found in the basal cells of the bronchial epithelium. Furthermore, immunohistochemistry for CK17 was analyzed in more detail on 10 G2 and 5 G3 SCC tumor samples. These staining results show a higher expression of CK17 in moderately differentiated G2 grade SCC tumors as compared to poorly differentiated G3 grade SCC tumors. HSP-47 was significantly overexpressed in SCC, but also in adenocarcinoma and large cell carcinoma as compared to bronchial epithelium. We conclude that CK6a and CK16 are potential biomarkers for SCC that CK17 expression refers to tumor load and is a potential SCC tumor grade marker.

We claim that using specific monoclonal antibodies we can detect CK6a and CK16 in serum samples and use the titer to discriminate SCC from other NSCLC (Adenocarcnioma and large cell carcinoma) and healthy controls. The SCC subtype of NSCLC can be characterized by the expression of at least one of the cytokeratins selected from the group consisting of CK4, CK5, CK6, CK10, CK13, CK14, CK15, CK16, CK17 and CK19 in the absence of NCAM 180, CK20 and CK7. CK17 serum titer can additionally be used to refer to tumor load and to discriminate G2 and G3 grade SCC tumor stage.

### Example 5

### Combination of a Selection of Specific Biomarkers Can be Used for Subtyping and Staging of Lung Cancer in a Serum Assay

We claim that using the combination of set of specific monoclonal antibodies for the antigen, serum antigen levels can be detected for NCAM exon 18-antigen, NCAM, NSP and various cytokines. In the following table the various antigens with the respective couples of specific capture and detector antibodies are shown.

| | | Monoclonal antibodies | |
|---|---|---|---|
| | | Capture | Detection |
| | Target Antigen | antibody | antibody |
| SCLC | NCAM Exon 18 | MUMI21B2 | MUM1 |
| | | | MUM4 |
| | | | MUM6 |
| | | RNL-1 | MUMI21B2 |
| NE differ- | NCAM | 123C3 | RNL-1 |
| entiation | NSP-A | RNL-2 | RNL-2 |
| | | RNL-3 | RNL-1 |
| SCC | Cytokeratin | CK6/CK16/CK17 | CK6/CK16/CK17 |

We claim that serum detection of NCAM exon 18-antigen titers can be used to discriminate SCLC from NSCLC.

We claim that serum detection of a panel of the tumor antigens consisting of NCAM, NCAM exon 18-antigen, NSP (in particular NSP-A) and two or more cytokeratin antigen (with in particular CK6, CK16 and CK17; can be used to discriminate tumor patients from non-tumor patients like COPD, to discriminate SCLC from NSCLC, and to specify within said tumors, tumors with or without Neuroendocrine (NE) origin, and squamous cell carcinomas (SCC). In said panel NCAM exon 18-antigen allows to discriminate SCLC from NSCLC and to discriminate SCLC from patients with NE tumors.

In said panel, serum detection of NCAM and NSP antigen titers can be used to discriminate lung tumors with NE characteristics from lung tumors with no NE origin.

In said panel, the detection of a specific selection of cytokeratin antigens being CK6a, CK16 and CK17 can be used to specifically subtype SCC within NSCLC.

A diagnostic guideline on how to use the lung cancer subtype specific biomarkers of the present invention is shown in FIG. 7.

### REFERENCE LIST

¹ Senden N H et al, Cluster-10 lung-cancer antibodies recognize NSPs, novel neuroendocrine proteins associated with membranes of the endoplasmic reticulum. Int. J. Cancer Suppl., 8, 84-88, 1994
² van de Velde H J K et al, NSP-encoded reticulon, neuroendocrine proteins of a novel gene family associated with membranes of the endoplasmic reticulum. Journal of Cell Science, 107, 2403-2416, 1994.
³ van de Velde H J K et al, NSP-encoded reticulons are neuroendocrine markers of a novel category in human lung cancer diagnosis. Cancer Research, 54, 4769-4776, 1994.
⁴ Senden N H M et al, A comparison of NSP-reticulons with conventional neuroendocrine markers in immunophenotyping of lung cancers. Journal of Pathology, 182, 13-21, 1997
⁵ Senden, N H M, Neuroendocrine-specific protein (NSP)-reticulons as independent markers for non-small cell lung cancer with neuroendocrine differentiation. An in vitro histochemical study. Histochem Cell Biol., 108, 155-165, 1997
⁶ Poschmann G et al, Identification of proteome differences between squamous cell carcinoma of the lung and bronchiale pithelium. Molecular and Cellular Proteomics, January 27, M800422-MCP200, 2009.
⁷ Sitek B et al, Application of fluorescence difference gel electrophoresis saturation labelling for the analysis of microdissected precursor lesions of pancreatic ductal adenocarcinoma. Proteomics 5, 2665-2679, 2005.

1-32. (canceled)

33. A method of detecting, diagnosing, subtyping, staging, or determining the degree of heterogeneity of lung cancer in a subject comprising:
(1) obtaining a sample from said subject;
(2) determining the expression or antibody titers of NCAM 180 or the NCAM splice variant containing NCAM exon 18-antigen region; and
(3) determining the expression of at least one tumor marker gene selected from the group consisting of NCAM splice variants NCAM 120 or NCAM 140; a cytokeratin (CK); Neuronendocrine specific protein (NSP)-reticulons (RTN1); and Heat Shock Protein-47 (HSP47) or antibody titers against at least one of said genes or proteins; wherein the expression of said genes or the presence or absence of said proteins allows detection, diagnosis, subtyping, staging, or determination of the degree of heterogeneity of the lung cancer in said subject.

34. A method according to claim 33, wherein the cytokeratin (CK) is selected from the group consisting of CK4, CK5, CK6 (CK6a and CK6b), CK7, CK8, CK10, CK13, CK14, CK15, CK16, CK17, CK18, CK19 and CK20.

35. A method according to claim 34, wherein the expression or presence of at least two of said cytokeratin genes or proteins is determined.

36. A method according to claim 35, wherein the expression or presence of at least two of said cytokeratin genes or proteins is determined using antibodies that specifically bind to the proteins encoded by said genes.

37. A method according to claim 33 wherein the expression of NCAM 180, the NCAM splice variant expressing the NCAM exon 18, CK4, CK5, CK6, CK10, CK13, CK14, CK15, CK16, CK17 and CK20 are used to differentiate SCLC from NSCLC, wherein SCLC is characterized by the expression/detection of NCAM 180, CK8 and CK18 in the absence of CK4, CK5, CK6, CK10, CK13, CK14, CK15, CK16, CK17 and CK20; and NSCLC is characterized by the absence of NCAM 180 in the presence of CK19, CK6/CK16/CK17 or CK8/CK18.

38. A method according to claim 33 wherein the expression of NCAM 180, CK4, CK5, CK6, CK7, CK8, CK10, CK13, CK14, CK15, CK16, CK17, CK18, CK19 or CK20 are used to detect or diagnose NSCLC or to determine the differentiation or subtype of NSCLC's (adenocarcinoma versus squamous cell carcinoma), wherein adenocarcinoma differentiation or subtype of NSCLC is characterized by the expression of CK7, CK8, CK18 and CK19 in the absence of CK20, NCAM 180 and one or more of the cytokeratin genes selected from the group consisting of CK4, CK5, CK6, CK10, CK13, CK14, CK15, CK16 and CK17; and squamous cell carcinoma differentiation, subtype of NSCLC, or SSC staging is characterized by the expression of at least one of the cytokeratin genes selected from the group consisting of CK4, CK5, CK6, CK10, CK13, CK14, CK15, CK16, CK17 and CK19 in the absence of NCAM180, CK20 and CK7.

39. A method according to claim 33 wherein neuroendocrine differentiation of lung cancers is characterized by the expression of NCAM and at least one of the genes selected from the group NSP, SYPH, and CHGA.

40. A method according to claim 34 in which the expression of CK8, CK18 and CK20 can be used to diagnose neuroendocrine Merkel Cell carcinomas.

41. A method according to claim 33 wherein the sample is selected from the group consisting of blood, serum, plasma, urine, saliva, semen, breast exudates, cerebrospinal fluid, tears, sputum, mucous, lymph, pleural effusions, tumor tissue and bronchioalveolar lavages.

42. A method according to claim 33 wherein the antibody titers against at least one of the said genes/proteins in one of the said samples is determined by an immunoassay.

43. A method according to claim 33 further including using one or more of the tumor marker genes from step (3) to monitor the response of a subject to lung cancer treatment or for diagnosing, staging, or subtyping lung cancer in said subject.

44. A method according to claim 33 further including using one or more of the marker genes selected from the group consisting of NCAM, NCAM Exon 18, NSP, and two or more cytokeratins to image lung cancer in said subject.

45. An in vitro method for (early) diagnosis, subtyping, and determination of the differentiation of lung cancer in a subject, said method comprising:
(a) obtaining a sample from said subject; and
(b) determining the expression of a panel of tumor marker genes consisting of NCAM, NCAM Exon 18, Neuroendocrine specific protein (NSP) and two or more cytokeratin antigens; wherein the expression of said genes or the presence or absence of said protein or antigens provides for the detection, diagnosis, subtype or determination of the degree of heterogeneity or staging of the lung cancer in said subject.

46. A method according to claim 45 wherein the sample is selected from the group consisting of blood, serum, plasma, urine, saliva, semen, breast exudates, cerebrospinal fluid, tears, sputum, mucous, lymph, pleural effusions, tumor tissue and bronchioalveolar lavages.

47. A method according to claim 45, wherein the expression level of said tumor marker genes is determined at the protein level or the nucleic acid level.

48. A method according to claim 47 wherein the expression level is determined by chemiluminescence, absorbance, western blotting, microscopy, imaging, immunoassay, or hybridization assay.

49. A method according to claim 47 wherein said expression level is determined by an immunoassay selected from the group consisting of ELISA, IRMA, Evanescence, lateral flow, or immuno histo-cyto-chemistry.

50. A kit for the detection, subtyping, staging, or determination of the degree of heterogeneity of lung cancer, including both SCLC and NSCLC, in a subject, comprising one or more reagents to determine the expression of a tumor marker gene selected from the group consisting of NCAM splice variants NCAM 120 or NCAM 140; a cytokeratin (CK); Neuronendocrine specific protein (NSP)-reticulons (RTN1); and Heat Shock Protein-47 (HSP47).
